(19) United States
(12) Patent Application Publication
(10) Pub. No.: US 2012/0064091 A1
(43) Pub. Date: Mar. 15, 2012

(54) DYSFUNCTION OF THE MITOCHONDRIAL RESPIRATORY CHAIN, METHODS FOR DIAGNOSIS, TREATMENT AND FOLLOW-UP

(75) Inventors: Anu Wartiovaara, Helsinki (FI); Henna Tyynismaa, Espoo (FI); Nuno Das Neves Raimundo, Portalegre (PT)

(73) Assignee: University of Helsinki, University of Helsinki (FI)

(21) Appl. No.: 13/259,125

(22) PCT Filed: Mar. 23, 2010
(86) PCT No.: PCT/FI2010/050223

§ 371 Date: Oct. 12, 2011

Related U.S. Application Data
(60) Provisional application No. 61/162,975, filed on Mar. 24, 2009.

(51) Int. Cl. A61K 39/395; A61K 38/18; H01J 49/26; A61P 21/00; A61P 9/00; A61P 1/16; G01N 33/566; A61P 25/00

(52) U.S. Cl. 424/158.1; 435/7.92; 436/501; 424/172.1; 514/9.1; 250/282

(57) ABSTRACT

The invention relates to a method, of assessing whether a subject is affected with or at risk for developing a dysfunction of the mitochondrial respiratory chain, the method comprising determining the concentration of fibroblast growth factor 21 (FGF21) in a biological sample from the subject, and comparing it to the concentration of FGF21 in a biological sample from at least one normal control, wherein an increase in the concentration of FGF21 in the biological sample from the subject when compared to the concentration of FGF21 in the biological sample from at least one normal control is indicative of occurrence of the dysfunction of the mitochondrial respiratory chain in said subject, or of risk for developing said dysfunction. The invention also relates to a method for follow-up of a dysfunction of the mitochondrial respiratory chain in a subject diagnosed with but not being treated for said dysfunction, to a method for treating a subject having a dysfunction of the mitochondrial respiratory chain, to a method for determining whether a dysfunction of the mitochondrial respiratory chain in a subject diagnosed with and being treated for said dysfunction is responding to the treatment, and for selecting patients for clinical trials.

The invention relates to a method for treating a subject having a dysfunction of the mitochondrial respiratory chain and to method for diagnosis and follow up.

### BACKGROUND

Progressive dysfunction of the mitochondrial respiratory chain (RC) is a common cause of adult-onset myopathies and neurodegenerative disorders. The morphological hallmark of RC deficiency in skeletal muscle is the presence of cytochrome c oxidase (COX) deficient muscle fibers. Secondary RC dysfunction with COX-negative muscle fibers is common in degenerative or inflammatory diseases, such as inclusion body myositis (Oldfors et al., 2006). However, decline of RC function also occurs in the normal population: after 50-60 years of age, COX-negative segments with mitochondrial proliferation are found in about 5% of muscle fibers, reaching up to 30% at 90 years of age (Bua et al., 2006). In spite of the common occurrence, the physiological consequences of progressive mitochondrial RC dysfunction are not well known.

Familial progressive external ophthalmoplegia (PEO) with multiple mtDNA deletions (Suomalainen et al., 1992; Zeviani et al., 1989) is a typical example of a progressive adult-onset RC deficiency. The present inventors and others have reported mutations in four genes underlying the trait: adenine-nucleotide translocator (ANT1), DNA polymerase gamma (POLG1 and POLG2) and DNA helicase Twinkle (Kaukonen et al., 2000; Longley et al., 2006; Spelbrink et al., 2001; Van Goethem et al., 2001). In PEO, the primary nuclear gene mutation induces secondary deletion formation in mtDNA. The mtDNA deletions accumulate with age and the disease manifests when the mutant mtDNA exceeds a threshold that causes RC deficiency. Familial PEO patients typically have 5-20% COX-negative fibers in their muscle (Suomalainen et al., 1997).

### OBJECT OF THE INVENTION

An object of the present invention is to provide a method of assessing whether a subject is affected with or at risk for developing a dysfunction of the mitochondrial respiratory chain.

A further object of the invention is a method for follow-up of a dysfunction of the mitochondrial respiratory chain in a subject diagnosed with but not being treated for said dysfunction.

A further object of the invention is a method for treating a subject having a dysfunction of the mitochondrial respiratory chain.

A further object of the invention is a method for determining whether a dysfunction of the mitochondrial respiratory chain in a subject diagnosed with and being treated for said dysfunction is responding to the treatment, and for selecting patients for clinical trials.

### SUMMARY OF THE INVENTION

Cytochrome-c-oxidase (COX) deficient muscle fibers are typical for mitochondrial diseases, and occur in normal aging. However, physiological consequences of muscle COX-deficiency are poorly characterized. The present inventors studied the global expression pattern of skeletal muscle of mice with late-onset mitochondrial myopathy. The present inventors identified up-regulation of starvation response, with increased expression of a novel fasting-induced hormone, FGF21. COX-negative muscle fibers specifically expressed this hormone, leading to increased serum FGF21 concentrations, induced lipolysis and to resistance to high-fat-diet-induced obesity, mimicking FGF21-overexpressing mice. FGF21 levels correlated with the presence of COX-negative fibers both in mice and in mitochondrial myopathy patients. Data obtained by the inventors shows that COX-deficiency in single muscle fibers is interpreted as a state of starvation and initiates a global starvation response through FGF21. Serum FGF21 offers a novel biomarker for mitochondrial disease diagnosis and follow-up, presently based on muscle biopsy. This data has important implications for conditions with primary or secondary COX-deficiency.

### DETAILED DESCRIPTION OF THE INVENTION

Based on the foregoing, the present invention provides a method of assessing whether a subject is affected with or at risk for developing a dysfunction of the mitochondrial respiratory chain, the method comprising determining the concentration of fibroblast growth factor 21 (FGF21) in a biological sample from the subject, and comparing it to the concentration of FGF21 in a biological sample from at least one normal control, wherein an increase in the concentration of FGF21 in the biological sample from the subject when compared to the concentration of FGF21 in the biological sample from at least one normal control is indicative of occurrence of the dysfunction of the mitochondrial respiratory chain in said subject, or of risk for developing said dysfunction.

According to one preferable embodiment of the method the dysfunction of the mitochondrial respiratory chain is of a type manifesting with muscle-related or brain-related or heart related or liver-related symptoms.

According to another preferable embodiment of the method the dysfunction of the mitochondrial respiratory chain is of a type previously known to involve cytochrome-c-oxidase negative muscle fibers.

Suitably the biological sample is selected from the group consisting of blood, serum, plasma, urine, cerebrospinal fluid, and other bodily fluids.

Preferably the concentration of FGF21 in the biological sample is determined by a detection method selected from the group consisting of ELISA, RIA and other immuno-detection based methods, other methods based on direct protein identification such as mass spectrometric analysis, and any other suitable detection methods.

According to another preferable embodiment of the method the extent of the increase in said concentration of FGF21, as compared to the normal control, correlates with the severity of the mitochondrial alterations in the muscles of said subject.

The present invention also provides a method for follow-up of a dysfunction of the mitochondrial respiratory chain in a subject diagnosed with but not being treated for said dysfunction, the method comprising the steps of:
a. obtaining a biological sample from said subject;
b. determining the concentration of fibroblast growth factor 21 (FGF21) in the biological sample; and
c. evaluating the concentration of FGF21, wherein an increase in the concentration of FGF21 in the biological sample from said subject when compared to the concentration of FGF21 in a biological sample taken from the same subject at an earlier time point is indicative of a progression of said dysfunction, whereas a decrease is indicative of a regression of said dysfunction.

According to one preferable embodiment of the method the dysfunction of the mitochondrial respiratory chain is of a type manifesting with muscle-related or brain-related or heart related or liver-related symptoms.

According to another preferable embodiment of the method the dysfunction of the mitochondrial respiratory chain is of a type previously known to involve cytochrome-c-oxidase negative muscle fibers.

Suitably the biological sample is selected from the group consisting of blood, serum, plasma, urine, cerebrospinal fluid, and other bodily fluids.

According to one preferable embodiment of the method the concentration of FGF21 in the biological sample is determined by a detection method selected from the group consisting of ELISA, RIA and other immuno-detection based methods, other methods based on direct protein identification such as mass spectrometric analysis, and any other suitable detection methods.

The present invention further provides a method for treating a subject having a dysfunction of the mitochondrial respiratory chain, wherein an agent inhibiting fibroblast growth factor 21 (FGF21) or inhibiting its receptor is administered to said subject.

According to one preferable embodiment of the method said agent inhibiting FGF21 or inhibiting its receptor is an antibody or a chemical inhibitor compound.

According to another preferable embodiment of the method the dysfunction of the mitochondrial respiratory chain is of a type manifesting with muscle-related or brain-related or heart related or liver-related symptoms.

According to another preferable embodiment of the method the dysfunction of the mitochondrial respiratory chain is of a type previously known to involve cytochrome-c-oxidase negative muscle fibers.

The present invention provides also a method for treating a subject having a dysfunction of the mitochondrial respiratory chain, wherein an agent mimicking or stimulating fibroblast growth factor 21 (FGF21) or its receptor, or in vitro produced recombinant FGF21, is administered to said subject.

According to a preferable embodiment of the method the dysfunction of the mitochondrial respiratory chain is of a type manifesting with muscle-related or brain-related or heart related or liver-related symptoms.

According to another preferable embodiment of the method the dysfunction of the mitochondrial respiratory chain is of a type previously known to involve cytochrome-c-oxidase negative muscle fibers.

Still, the present invention provides a method for determining whether a dysfunction of the mitochondrial respiratory chain in a subject diagnosed with and being treated for said dysfunction is responding to the treatment, and for selecting patients for clinical trials, the method comprising the steps of:
a. obtaining a biological sample from said subject;
b. determining the concentration of fibroblast growth factor 21 (FGF21) in the biological sample; and
c. evaluating the concentration of FGF21, wherein a decrease in the concentration of FGF21 in the biological sample from said subject when compared to the concentration of FGF21 in a biological sample taken from the same subject at an earlier time point is indicative of a positive response to the treatment, whereas an increase is indicative of a negative response to the treatment or non-responsiveness to the treatment.

According to a preferable embodiment of the method the dysfunction of the mitochondrial respiratory chain is of a type manifesting with muscle-related or brain-related or heart related or liver-related symptoms.

According to another preferable embodiment of the method the dysfunction of the mitochondrial respiratory chain is of a type previously known to involve cytochrome-c-oxidase negative muscle fibers.

Suitably the biological sample is selected from the group consisting of blood, serum, plasma, urine, cerebrospinal fluid, and other bodily fluids.

According to further preferable embodiment of the method the concentration of FGF21 in the biological sample is determined by a detection method selected from the group consisting of ELISA, RIA and other immuno-detection based methods, other methods based on direct protein identification such as mass spectrometric analysis, and any other suitable detection methods.

According to a preferable embodiment of the method, the extent of the decrease in said concentration of FGF21 as compared to the concentration at earlier time point correlates with the effectiveness of the treatment.

The inventors made a mouse model for PEO by expressing a dominant PEO mutation, a 13-amino-acid duplication in the mitochondrial replication helicase Twinkle, under ubiquitous beta-actin promoter (Tyynismaa et al., 2005). At one year of age, the mice presented with COX-deficient muscle fibers with numerous, enlarged and morphologically abnormal mitochondria, as well as induced autophagy of mitochondria. The mice had progressive accumulation of multiple mtDNA deletions in the muscle—hence called the Deletor—but had a normal lifespan. The Deletor mouse replicates the histological, genetic and biochemical features of PEO disease and was here used as a valuable tool to study pathogenesis of subtle progressive RC dysfunction of adult age. The inventors show here that RC deficiency initiates a global starvation response in mice and humans, through the newly identified hormone fibroblast growth factor 21 (FGF21) (Badman et al., 2007; Inagaki et al., 2007), secreted by the COX-negative muscle fibers.

Deletor mice manifest progressive mitochondrial myopathy at 12 months of age (Tyynismaa et al., 2005). The inventors performed gene expression profiling of quadriceps femoris muscle of 20-24 month-old Deletor mice. Most of the detected gene expression differences between the Deletor and control mice were fairly small, which is in agreement with the subtle progression and chronic nature of the Deletor phenotype. The most up- and down-regulated transcripts are listed in Table 1. All transcripts that filled the p-value criterion of <0.05 for being differentially expressed between the Deletor and control mice were subjected to Ingenuity Pathway Analysis to identify significantly altered pathways in our disease model. The advantage of the pathway analysis is that it can utilize information of many subtle but significant gene expression alterations at the same time, when considering their functional relationships. The most significantly induced pathway was the PI3K/AKT pathway (Table 1 and FIG. 1I), which included the down-regulation of mTOR (mammalian target of rapamycin) (Table S1).

Table 1. Upper panel: the six most up-regulated and down-regulated transcripts of skeletal muscle of Deletor mice in the microarray experiment, compared to controls. Lower panel: the ten most significantly altered pathways; transcript data analyzed with the Ingenuity Pathway Analysis. See Supplementary Table 1 for the list of differentially expressed genes in each pathway.

TABLE 1

| | | Fold | |
|---|---|---|---|
| Gene | Description | Change | p-value |
| MTHFD2 | Methyltetrahydrofolate dehydrogenase 2 | +4.49 | 0.024 |
| FGF21 | Fibroblast growth factor 21 | +3.70 | 0.017 |
| SFPQ | Splicing factor proline/glutamine-rich | +3.17 | 0.003 |
| MAP4 | Microtubule-associated protein 4 | +3.13 | 0.011 |
| CUL5 | Cullin 5 | +2.55 | 0.024 |
| TPR | Translocated promoter region | +2.51 | 0.019 |
| PER2 | Period homolog 2 | −1.84 | 0.015 |
| SOCS2 | Suppressor of cytokine signaling 2 | −1.69 | 0.009 |
| GPR90 | G protein-coupled receptor | −1.67 | 0.041 |
| GNA13 | Guanine nucleotide binding protein 13 | −1.60 | 0.001 |
| PLOD2 | Procollagen-lysine, 2-oxoglutarate 5- | −1.57 | 0.001 |
| | dioxygenase 2 | | |
| ACO1 | Aconitase 1 | −1.56 | 0.050 |

| | | Altered | | |
|---|---|---|---|---|
| | | genes/ | Up-re- | Down-re- |
| | | Genes in | gulated | gulated |
| Pathway | p-value | pathway | genes | genes |
| PI3K/AKT Signaling | 2.9 × 10⁻⁶ | 16/124 | 13 | 3 |
| Integrin Signaling | 2.8 × 10⁻⁴ | 187192 | 15 | 3 |
| Axonal Guidance Signaling | 5.0 × 10⁻⁴ | 27/386 | 18 | 9 |
| Tight Junction Signaling | 5.1 × 10⁻⁴ | 15/159 | 9 | 6 |
| PTEN Signaling | 1.6 × 10⁻³ | 10/92 | 10 | 0 |
| NRF2-mediated Oxidative | 1.9 × 10⁻³ | 15/180 | 13 | 2 |
| Stress Response | | | | |
| Nitric Oxide Signaling in | 2.3 × 10⁻³ | 8/85 | 8 | 0 |
| the Cardiovascular System | | | | |
| B Cell Receptor Signaling | 2.6 × 10⁻³ | 13/148 | 11 | 2 |
| Leukocyte Extravasation | 2.5 × 10⁻³ | 15/191 | 9 | 6 |
| Signaling | | | | |
| Alanine and Aspartate | 4.4 × 10⁻³ | 6/87 | 3 | 3 |
| Metabolism | | | | |

Supplementary Table 1 (Table S1).

| | | | | |
|---|---|---|---|---|
| Name | Description | Affymetrix | Fold Change | p-value |

PI3K/AKT signaling

| | | | | |
|---|---|---|---|---|
| AKT3 | v-akt murine thymoma viral oncogene homolog 3 (protein kinase B. gamma) | 1435260_at | 1.213 | 0.034 |
| CDKN1A | cyclin-dependent kinase inhibitor 1A (p21. Cip1) | 1424638_at | 1.704 | 0.008 |
| CTNNB1 | catenin (cadherin-associated protein). beta 1. 88kDa | 1430533_a_at | 1.291 | 0.025 |
| FOXO1 | forkhead box O1 | 1416981_at | 1.148 | 0.039 |
| FRAP1 | FK506 binding protein 12-rapamycin associated protein 1 | 1459625_at | −1.168 | 0.018 |
| GSK3B | glycogen synthase kinase 3 beta | 1437001_at | 1.851 | 0.048 |
| HSP90AA1 | heat shock protein 90kDa alpha (cytosolic). class A member 1 | 1437497_a_at | 1.861 | 0.017 |
| HSP90AB1 | heat shock protein 90kDa alpha (cytosolic). class B member 1 | 1416365_at | 1.414 | 0.047 |
| IKBKG | inhibitor of kappa light polypeptide gene enhancer in B-cells. kinase gamma | 1435646_at | 1.202 | 0.016 |
| PIK3CA | phosphoinositide-3-kinase. catalytic. alpha polypeptide | 1453134_at | 1.248 | 0.018 |
| PIK3R1 | phosphoinositide-3-kinase. regulatory subunit 1 (alpha) | 1425514_at | 1.334 | 0.044 |
| PPP2CB | protein phosphatase 2 (formerly 2A). catalytic subunit. beta isoform | 1431341_at | −1.307 | 0.028 |
| PPP2R2A | protein phosphatase 2 (formerly 2A). regulatory subunit B. alpha isoform | 1453260_a_at | 1.392 | 0.038 |
| PPP2R5D | protein phosphatase 2. regulatory subunit B′. delta isoform | 1450560_a_at | −1.279 | 0.003 |
| PTEN | phosphatase and tensin homolog (mutated in multiple advanced cancers 1) | 1422553_at | 1.617 | 0.029 |
| YWHAE | tyrosine 3-monooxygenase/tryptophan 5-monooxygenase activation protein. epsilon polypeptide | 1426384_a_at | 1.154 | 0.029 |

Integrin signaling

| | | | | |
|---|---|---|---|---|
| ACTG2 | actin. gamma 2. smooth muscle. enteric | 1422340_a_at | −1.194 | 0.017 |
| AKT3 | v-akt murine thymoma viral oncogene homolog 3 (protein kinase B. gamma) | 1435260_at | 1.213 | 0.034 |
| ARPC2 | actin related protein 2/3 complex. subunit 2. 34kDa | 1437148_at | 1.088 | 0.036 |
| ARPC5L | actin related protein 2/3 complex. subunit 5-like | 1452044_at | 1.199 | 0.024 |
| BCAR1 | breast cancer anti-estrogen resistance 1 | 1439388_s_at | 1.207 | 0.014 |
| BRAF | v-raf murine sarcoma viral oncogene homolog B1 | 1442749_at | 1.301 | 0.002 |
| GIT1 | G protein-coupled receptor kinase interactor 1 | 1454759_at | 1.324 | 0.042 |
| GSK3B | glycogen synthase kinase 3 beta | 1437001_at | 1.851 | 0.048 |
| ITGA7 | integrin. alpha 7 | 1418393_a_at | 1.326 | 0.04 |
| PIK3CA | phosphoinositide-3-kinase. catalytic. alpha polypeptide | 1453134_at | 1.248 | 0.018 |
| PIK3R1 | phosphoinositide-3-kinase. regulatory subunit 1 (alpha) | 1425514_at | 1.334 | 0.044 |
| PLCG2 | phospholipase C. gamma 2 (phosphatidylinositol-specific) | 1426926_at | −1.178 | 0.04 |
| PTEN | phosphatase and tensin homolog (mutated in multiple advanced cancers 1) | 1422553_at | 1.617 | 0.029 |
| RHOJ | ras homolog gene family. member J | 1444982_at | 1.144 | 0.024 |
| RHOU | ras homolog gene family. member U | 1449028_at | 1.537 | 0.016 |
| TSPAN3 | tetraspanin 3 | 1416009_at | −1.24 | 0.011 |
| TTN | titin | 1444638_at | 2.157 | 0.011 |
| WASL | Wiskott-Aldrich syndrome-like | 1426777_a_at | 1.498 | 0.023 |

Axonal guidance signaling

| | | | | |
|---|---|---|---|---|
| ADAM8 | ADAM metallopeptidase domain 8 | 1416871_at | −1.252 | 0.044 |
| ADAM19 | ADAM metallopeptidase domain 19 (meltrin beta) | 1418402_at | 1.192 | 0.038 |
| AKT3 | v-akt murine thymoma viral oncogene homolog 3 (protein kinase B. gamma) | 1435260_at | 1.213 | 0.034 |
| ARPC2 | actin related protein 2/3 complex. subunit 2. 34kDa | 1437148_at | 1.088 | 0.036 |
| ARPC5L | actin related protein 2/3 complex. subunit 5-like | 1452044_at | 1.199 | 0.024 |
| BCAR1 | breast cancer anti-estrogen resistance 1 | 1439388_s_at | 1.207 | 0.014 |
| GIT1 | G protein-coupled receptor kinase interactor 1 | 1454759_at | 1.324 | 0.042 |
| GLI1 | glioma-associated oncogene homolog 1 (zinc finger protein) | 1449058_at | −1.336 | 0.028 |
| GNA13 | guanine nucleotide binding protein (G protein). alpha 13 | 1422555_s_at | 1.437 | 0.036 |
| GNAI2 | guanine nucleotide binding protein (G protein). alpha inhibiting activity polypeptide 2 | 1435652_a_at | −1.154 | 0.016 |
| GNAI3 | guanine nucleotide binding protein (G protein). alpha inhibiting activity polypeptide 3 | 1437225_x_at | −1.602 | 0.001 |
| GSK3B | glycogen synthase kinase 3 beta | 1437001_at | 1.851 | 0.048 |
| MYL4 | myosin. light chain 4. alkali; atrial. embryonic | 1422580_at | 2.064 | 0.007 |
| NFAT5 | nuclear factor of activated T-cells 5. tonicity-responsive | 1439805_at | 1.746 | 0.002 |
| NFATC1 | nuclear factor of activated T-cells. cytoplasmic. calcineurin-dependent 1 | 1417621_at | 1.276 | 0 |
| NRP1 | neuropilin 1 | 1448944_at | 1.165 | 0.002 |
| NRP2 | neuropilin 2 | 1426528_at | −1.305 | 0.026 |
| NTF3 | neurotrophin 3 | 1434802_s_at | 1.112 | 0.035 |
| NTRK3 | neurotrophic tyrosine kinase. receptor. type 3 | 1422329_a_at | −1.26 | 0.014 |
| PIK3CA | phosphoinositide-3-kinase. catalytic. alpha polypeptide | 1453134_at | 1.248 | 0.018 |
| PIK3R1 | phosphoinositide-3-kinase. regulatory subunit 1 (alpha) | 1425514_at | 1.334 | 0.044 |
| PLXNA3 | plexin A3 | 1420996_at | −1.277 | 0.023 |
| RTN4 | reticulon 4 | 1437224_at | 1.500 | 0.044 |
| SEMA4B | semaphorin 4B | 1455678_at | −1.118 | 0.043 |
| SEMA5A | semaphorin 5A | 1437422_at | 1.419 | 0.01 |
| WASL | Wiskott-Aldrich syndrome-like | 1426777_a_at | 1.498 | 0.023 |
| WNT5B | wingless-type MMTV integration site family. member 5B | 1433814_at | −1.087 | 0.048 |

Tight junction signaling

| | | | | |
|---|---|---|---|---|
| ACTG2 | actin. gamma 2. smooth muscle. enteric | 1422340_a_at | −1.194 | 0.017 |
| AKT3 | v-akt murine thymoma viral oncogene homolog 3 (protein kinase B. gamma) | 1435260_at | 1.213 | 0.034 |
| ARHGEF2 | rho/rac guanine nucleotide exchange factor (GEF) 2 | 1421043_s_at | 1.480 | 0.008 |
| CLDN1 | claudin 1 | 1450014_at | −1.269 | 0.045 |
| CLDN15 | claudin 15 | 1418920_at | −1.166 | 0.04 |
| CPSF2 | cleavage and polyadenylation specific factor 2. 100kDa | 1420937_at | 1.213 | 0.024 |
| CSTF2 | cleavage stimulation factor. 3′ pre-RNA. subunit 2. 64kDa | 1455523_at | −1.331 | 0.048 |
| CTNNB1 | catenin (cadherin-associated protein). beta 1. 88kDa | 1430533_a_at | 1.291 | 0.025 |
| MLLT4 | myeloid/lymphoid or mixed-lineage leukemia (trithorax homolog. Drosophila); translocated to. 4 | 1436303_at | 1.356 | 0.016 |
| MYL4 | myosin. light chain 4. alkali; atrial. embryonic | 1422580_at | 2.064 | 0.007 |
| PPP2CB | protein phosphatase 2 (formerly 2A). catalytic subunit. beta isoform | 1431341_at | −1.307 | 0.028 |
| PPP2R2A | protein phosphatase 2 (formerly 2A). regulatory subunit B. alpha isoform | 1453260_a_at | 1.392 | 0.038 |
| PPP2R5D | protein phosphatase 2. regulatory subunit B′. delta isoform | 1450560_a_at | −1.279 | 0.003 |
| PTEN | phosphatase and tensin homolog (mutated in multiple advanced cancers 1) | 1422553_at | 1.617 | 0.029 |
| SYMPK | symplekin | 1428673_at | 1.392 | 0.045 |

PTEN signaling

| | | | | |
|---|---|---|---|---|
| AKT3 | v-akt murine thymoma viral oncogene homolog 3 (protein kinase B. gamma) | 1435260_at | 1.213 | 0.034 |
| BCAR1 | breast cancer anti-estrogen resistance 1 | 1439388_s_at | 1.207 | 0.014 |
| BCL2L11 | BCL2-like 11 (apoptosis facilitator) | 1456005_a_at | 1.527 | 0.039 |
| CDKN1A | cyclin-clependent kinase inhibitor 1A (p21. Cip1) | 1424638_at | 1.704 | 0.008 |
| FOXO1 | forkhead box O1 | 1416981_at | 1.148 | 0.039 |
| GSK3B | glycogen synthase kinase 3 beta | 1437001_at | 1.851 | 0.048 |
| IKBKG | inhibitor of kappa light polypeptide gene enhancer in B-cells. kinase gamma | 1435646_at | 1.202 | 0.016 |
| PIK3CA | phosphoinositide-3-kinase. catalytic. alpha polypeptide | 1453134_at | 1.248 | 0.018 |
| PIK3R1 | phosphoinositide-3-kinase. regulatory subunit 1 (alpha) | 1425514_at | 1.334 | 0.044 |
| PTEN | phosphatase and tensin homolog (mutated in multiple advanced cancers 1) | 1422553_at | 1.617 | 0.029 |

NRF2-mediated oxidative stress response

| | | | | |
|---|---|---|---|---|
| ACTG2 | actin. gamma 2. smooth muscle. enteric | 1422340_a_at | −1.194 | 0.017 |
| DNAJB14 | DnaJ (Hsp40) homolog. subfamily B. member 14 | 1430561_at | 1.956 | 0.023 |
| DNAJC1 | DnaJ (Hsp40) homolog. subfamily C. member 1 | 1420500_at | 1.462 | 0.023 |
| GSK3B | glycogen synthase kinase 3 beta | 1437001_at | 1.851 | 0.048 |
| GSTA5 | glutathione S-transferase A5 | 1421041_s_at | 1.319 | 0.025 |
| GSTM4 | glutathione S-transferase M4 | 1424835_at | −1.194 | 0.037 |
| JUNB | jun B proto-oncogene | 1415899_at | 1.590 | 0.004 |
| MAF | v-maf musculoaponeurotic fibrosarcoma oncogene homolog (avian) | 1444073_at | 1.349 | 0.014 |
| MAFG | v-maf musculoaponeurotic fibrosarcoma oncogene homolog G (avian) | 1448916_at | 1.230 | 0.023 |
| MAP3K7 | mitogen-activated protein kinase kinase kinase 7 | 1425795_a_at | 1.321 | 0.025 |
| PIK3CA | phosphoinositide-3-kinase. catalytic. alpha polypeptide | 1453134_at | 1.248 | 0.018 |
| PIK3R1 | phosphoinositide-3-kinase. regulatory subunit 1 (alpha) | 1425514_at | 1.334 | 0.044 |
| SCARB1 | scavenger receptor class B. member 1 | 1437378_x_at | 1.411 | 0.039 |
| SOD2 | superoxide dismutase 2. mitochondrial | 1454976_at | 1.317 | 0.028 |
| TXNRD1 | thioredoxin reductase 1 | 1421529_a_at | 1.262 | 0.048 |

Nitric oxide signaling in the cardiovascular system

| | | | | |
|---|---|---|---|---|
| AKT3 | v-akt murine thymoma viral oncogene homolog 3 (protein kinase B. gamma) | 1435260_at | 1.213 | 0.034 |
| CALM3 | calmodulin 3 (phosphorylase kinase. delta) | 1438826_x_at | 1.052 | 0.043 |
| FLT1 | fms-related tyrosine kinase 1 (vascular endothelial growth factor/vascular | 1419300_at | 1.397 | 0.007 |
| | permeability factor receptor) | | | |
| GUCY1A3 | guanylate cyclase 1. soluble. alpha 3 | 1434141_at | 1.498 | 0.042 |
| HSP90AA1 | heat shock protein 90kDa alpha (cytosolic). class A member 1 | 1437497_a_at | 1.861 | 0.017 |
| HSP90AB1 | heat shock protein 90kDa alpha (cytosolic). class B member 1 | 1416365_at | 1.414 | 0.047 |
| PIK3CA | phosphoinositide-3-kinase. catalytic. alpha polypeptide | 1453134_at | 1.248 | 0.018 |
| PIK3R1 | phosphoinositide-3-kinase. regulatory subunit 1 (alpha) | 1425514_at | 1.334 | 0.044 |

B cell receptor signaling

| | | | | |
|---|---|---|---|---|
| AKT3 | v-akt murine thymoma viral oncogene homolog 3 (protein kinase B. gamma) | 1435260_at | 1.213 | 0.034 |
| BCL6 | B-cell CLL/lymphoma 6 (zinc finger protein 51) | 1450381_a_at | 1.430 | 0.025 |
| CALM3 | calmodulin 3 (phosphorylase kinase. delta) | 1438826_x_at | 1.052 | 0.043 |
| FRAP1 | FK506 binding protein 12-rapamycin associated protein 1 | 1459625_at | −1.168 | 0.018 |
| GSK3B | glycogen synthase kinase 3 beta | 1437001_at | 1.851 | 0.048 |
| IKBKG | inhibitor of kappa light polypeptide gene enhancer in B-cells. kinase gamma | 1435646_at | 1.202 | 0.016 |
| MAP3K7 | mitogen-activated protein kinase kinase kinase 7 | 1425795_a_at | 1.321 | 0.025 |
| NFAT5 | nuclear factor of activated T-cells 5. tonicity-responsive | 1439805_at | 1.746 | 0.002 |
| NFATC1 | nuclear factor of activated T-cells. cytoplasmic. calcineurin-dependent 1 | 1417621_at | 1.276 | 0 |
| PIK3CA | phosphoinositide-3-kinase. catalytic. alpha polypeptide | 1453134_at | 1.248 | 0.018 |
| PIK3R1 | phosphoinositide-3-kinase. regulatory subunit 1 (alpha) | 1425514_at | 1.334 | 0.044 |
| PLCG2 | phospholipase C. gamma 2 (phosphatidylinositol-specific) | 1426926_at | −1.178 | 0.04 |
| PTEN | phosphatase and tensin homolog (mutated in multiple advanced cancers 1) | 1422553_at | 1.617 | 0.029 |

Leukocyte extravasation signaling

| | | | | |
|---|---|---|---|---|
| ACTG2 | actin. gamma 2. smooth muscle. enteric | 1422340_a_at | −1.194 | 0.017 |
| BCAR1 | breast cancer anti-estrogen resistance 1 | 1439388_s_at | 1.207 | 0.014 |
| CDH5 | cadherin 5. type 2. VE-cadherin (vascular epithelium) | 1422047_at | 1.136 | 0.037 |
| CLDN1 | claudin 1 | 1450014_at | −1.269 | 0.045 |
| CLDN15 | claudin 15 | 1418920_at | −1.166 | 0.04 |
| CTNNB1 | catenin (cadherin-associated protein). beta 1. 88kDa | 1430533_a_at | 1.291 | 0.025 |
| EZR | ezrin | 1450850_at | 1.178 | 0.022 |
| GNAI2 | guanine nucleotide binding protein (G protein). alpha inhibiting activity polypeptide 2 | 1435652_a_at | −1.154 | 0.016 |
| GNAI3 | guanine nucleotide binding protein (G protein). alpha inhibiting activity polypeptide 3 | 1437225_x_at | −1.602 | 0.001 |
| MLLT4 | myeloid/lymphoid or mixed-lineage leukemia (trithorax homolog. Drosophila); translocated to. 4 | 1436303_at | 1.356 | 0.016 |
| PIK3CA | phosphoinositide-3-kinase. catalytic. alpha polypeptide | 1453134_at | 1.248 | 0.018 |
| PIK3R1 | phosphoinositide-3-kinase. regulatory subunit 1 (alpha) | 1425514_at | 1.334 | 0.044 |
| PLCG2 | phospholipase C. gamma 2 (phosphatidylinositol-specific) | 1426926_at | −1.178 | 0.04 |
| TIMP2 | TIMP metallopeptidase inhibitor 2 | 1420924_at | 1.300 | 0.001 |
| WASL | Wiskott-Aldrich syndrome-like | 1426777_a_at | 1.498 | 0.023 |

Alanine and aspartate metabolism

| | | | | |
|---|---|---|---|---|
| AARS | alanyl-tRNA synthetase | 1423685_at | 1.198 | 0.011 |
| CRAT | carnitine acetyltransferase | 1441919_x_at | −1.312 | 0.021 |
| DLAT | dihydrolipoamide S-acetyltransferase | 1426264_at | −1.105 | 0.025 |
| GPT2 | glutamic pyruvate transaminase (alanine aminotransferase) 2 | 1434542_at | 1.353 | 0.041 |
| NARS | asparaginyl-tRNA synthetase | 1428666_at | 1.099 | 0.006 |
| NARS2 | asparaginyl-tRNA synthetase 2. mitochondrial (putative) | 1442989_at | −1.108 | 0.024 |

### Metabolic Regulator FGF21 is Induced in Deletor Muscle and Plasma

Fibroblast growth factor 21 was the second-most up-regulated gene in the Deletor muscle (P=0.017) (Table 1). FGF21 was recently described as the ‘missing link in the biology of fasting’ (Reitman, 2007), a hormone being expressed and secreted by the liver and adipose tissue in starvation (Badman et al., 2007; Inagaki et al., 2007). The previous electron-microscopic studies carried out by the inventors showed that autophagy was activated to recycle damaged mitochondria in the Deletor muscle, and that myofibril structures had been degraded in those muscles as a sign of protein degradation and catabolism (Tyynismaa et al., 2005). FGF21 induction suggested that the Deletor muscle fibers, suffering from RC dysfunction, are in a starvation-like state. The FGF21 mRNA levels in Deletors followed closely the amount of COX-negative muscle fibers in the disease progression in both Deletor founder lines C and D (FIG. 1A). The mice over-expressing wild type Twinkle did not show FGF21 induction (P=0.46) Therefore, FGF21 induction was transgene insertion site independent and specific to the mitochondrial myopathy.

FGF21 protein has a predicted size of 23 kDa, but plasma samples of mice also show an isoform of 30 kDa, which was efficiently removed by a blocking peptide (Muise et al., 2008). In Deletor and control mouse muscle samples we also detected the two isoforms (FIG. 1B). Interestingly, the 30 kDa isoform, which has specifically been found to be induced in fasting (Muise et al., 2008), was significantly increased in the Deletor muscle (P=0.002) whereas the level of the 23 kDa protein was unaltered (FIG. 1B).

The Deletor muscle showed several FGF21-positive muscle fibers by immunohistochemistry (FIG. 1C). These fibers were always severely affected, SDH-positive, with mitochondrial accumulation (FIGS. 1D and 1E). In longitudinal paraffin sections, FGF21 immunoreactivity was punctate, in vesicles around the nuclei but also in the cytoplasm of the muscle fibers (FIG. 1F), similar to the ‘storage vesicles’ that have been described for glucose transporter Glut4 (Lauritzen et al., 2008). No FGF21 positivity was present in the controls (FIG. 1G). These results clearly indicate that single skeletal muscle fibers induce FGF21 expression upon RC deficiency.

The inventors then asked whether the muscle-derived FGF21 could be secreted. The inventors determined by radioimmunoassay that the Deletors had 3-fold higher levels of FGF21 in plasma than the controls (FIG. 1H, P-0.018). This indicated that FGF21 was secreted and was systemically available. To clarify the source of FGF21 in the serum, the inventors studied its expression in liver and adipose tissue, which are known to secrete FGF21 in lack of nutrition (Inagaki et al., 2007; Muise et al., 2008). These tissues did not show increased FGF21 expression levels (data not shown) indicating that the skeletal muscle was a likely source of the elevated plasma FGF21 in the Deletor mice.

FGF21 is Induced in the Muscle and Serum of Patients with Mitochondrial Myopathy

The inventors then asked whether FGF21 was induced in human patients with mitochondrial disease. Frozen muscle samples from three PEO patients, who carried the dominant Twinkle mutation homologous to the Deletor mice (Spelbrink et al., 2001; Suomalainen et al., 1992; Suomalainen et al., 1997), showed clear FGF21 induction in selective, COX-deficient and SDH-positive muscle fibers (FIGS. 3A, 3B and 3c), but healthy controls showed no FGF21-positivity (data not shown). Importantly, also human patients with mitochondrial myopathy [two PEO patients, two MELAS (mitochondrial encephalopathy, lactic acidosis and stroke-like episodes) patients] showed clearly increased FGF21 levels in their serum (FIG. 3D). The serum FGF21 in the two PEO patients and the MELAS patient 2 were in the range of 627-731 ng/ml whereas MELAS patient 1 had nearly 1880 ng/ml of FGF21 in serum. The two serum samples of healthy individuals that we tested had 59 and 52 ng/ml of FGF21. The same ELISA assay that we used here has been used to measure serum FGF21 in 17 healthy controls in a Czech study (mean 272.3±40.04 pg/ml) (Dostalova et al., 2008) and in 105 Chinese individuals with normal weight (median 208.7 pg/ml, interquartile range 94.4-325.7) (Zhang et al., 2008). These results suggest that RC deficiency in the human skeletal muscle results in elevated serum FGF21 levels.

### The Metabolic Consequences of Induced FGF21 in the Deletor Mice

Previously, transgenic hepatic over-expression of FGF21 in mice resulted in elevated plasma FGF21 concentrations, and at 9 months of age these mice weighed significantly less, had less fat in liver, had adipocytes of smaller size and were resistant to high-fat diet induced obesity (Kharitonenkov et al., 2005). As these consequences are consistently observed in animals following FGF21 administration (Kharitonenkov and Shanafelt, 2008), the inventors studied whether the skeletal muscle-derived FGF21 had the same metabolic effects. Indeed, the Deletors had a smaller adipocyte size compared to age-matched control mice (50% reduction in D line, P=0.03; 36% reduction in C line, P=0.13) (FIGS. 4A and 4B), which was not due to qualitative or quantitative defects in mtDNA of white adipose tissue (analyzed by quantitative and deletion-specific PCR, data not shown). The livers of Deletors showed a clearly reduced amount of lipid droplets (FIG. 4C), also suggesting utilization of lipids. If RC deficiency was indeed misinterpreted as starvation in skeletal muscle, FGF21 signal could possibly be compensated by excess nutrition. Therefore the inventors gave the Deletors and their control littermates a high-fat (HF) diet from the age of 3 months to 14 months. The food-consumption and the stool lipid-content of the Deletors was similar to controls (data not shown), but they were resistant to weight-gain (FIG. 4D). HF-fed Deletors showed lower muscle FGF21 expression than Deletors on normal diet (FIG. 4E, P=0.02), but the levels remained high compared to those of wild-type mice. Excess calories could thus partially compensate the RC-induced starvation signal. All these findings are consistent with a global effect of muscle-derived FGF21 on lipid metabolism, closely mimicking the consequences of liver-specific FGF21 overexpression. FGF21 has been proposed to be a potent insulin sensitizer (Kharitonenkov and Shanafelt, 2008; Kharitonenkov et al., 2007), to cause growth hormone resistance in mice, leading to growth inhibition and elevated growth hormone levels (Inagaki et al., 2008), as well as to reduce the circulating levels of thyroid hormones in obese mice (Coskun et al., 2008). However, in the mice used by the inventors, skeletal muscle-derived FGF21 in the serum did not cause significant alterations in fasted insulin levels, or in the glucose or insulin tolerance of the Deletors compared to control littermates (FIGS. 4F, 4G and 4H). Furthermore, growth hormone levels as well as serum TSH and T3 levels of the Deletors were not significantly different from those of wild-type littermates (FIG. 2).

### Muscular FGF21 is not Induced in Transient Depletion of Energy Stores

The inventors also tested whether FGF21 secretion is a general physiological response of the healthy skeletal muscle in various stress situations. FGF21 induction in the liver has previously been reported in mice, which are either fasted (Inagaki et al., 2007) or fed with ketogenic diet (Badman et al., 2007). The inventors could replicate the starvation-related induction in the liver after 24 h fasting period. However, in the same fasting conditions that induced FGF21 expression in the liver 200-fold, FGF21 expression was not significantly altered in the skeletal muscle (FIG. 5A). High-fat diet of four days led to 2.5-fold FGF21 induction in the skeletal muscle (P=0.06) but the induction was modest when compared to that in the liver (50-fold) (FIG. 5B). Twelve months of HF-diet, or strenuous treadmill-training for a 6-week-period did not induce FGF21 in the muscle of control mice (FIGS. 4E and 5C). These studies indicate that transient depletion of energy stores does not induce FGF21 expression in a healthy skeletal muscle.

Physiological consequences of late-onset respiratory chain defects are not known, although this group of disorders is among the most frequent causes of inherited metabolic diseases and similar changes accumulate also in normal aging. The inventors show here that upon RC deficiency, skeletal muscle interprets the deficient oxidative ATP production as a state of starvation. This state is signalled to the whole organism by secretion of fasting-induced hormone FGF21 from the affected, pseudo-starved, muscle fibers. The study carried out by the inventors shows that mouse and human skeletal muscle can act as an endocrine organ that can secrete FGF21, inducing changes in adipocytes and lipolysis, and therefore providing a mechanism to recruit fuel for muscle mitochondria (FIG. 6). Since the primary defect is in mitochondrial ATP production, the starvation signal is sustained, and can only partially be compensated by excess nutrients in high-fat diet.

The inventors show here that FGF21 expression levels correlated with the amount of COX-negative fibers in a mouse model with mitochondrial myopathy. The inventors noticed in an expression array dataset (GEO: GDS1065) from mitochondrial myopathy patient muscle samples that the two patients with the highest number of COX-deficient muscle fibers (≧28%) also showed increased FGF21 mRNA levels. The inventors found both muscle and serum FGF21 increased in patients with COX-negative fibers, and the serum level of the patient with the most severe disease (MELAS patient 1) was the highest. The results obtained show that FGF21 induction is a general response, secreted from skeletal muscle upon. RC deficiency in mice and men, and that FGF21 levels correlate with the severity of the deficiency.

Importantly, detection of serum FGF21 in human patients offers a novel diagnostic biomarker. Currently, invasive skeletal muscle biopsy is the golden standard when mitochondrial myopathy is suspected in a patient, and patients may undergo several muscle samplings upon disease progression. The results obtained by the inventors suggest that FGF21 levels could be measured as a first-line diagnostic blood test, as well as for follow-up of disease progression. FGF21 is a valuable novel new tool for less-invasive mitochondrial disease diagnosis.

RC deficiency is the first example of a physiological state to which skeletal muscle responds as an independent endocrine organ, inducing a global hormonal response. Muscle-secreted hormones have been speculated to exist, with a potential to act on adipose, hepatic or central nervous system tissues (Izumiya et al., 2008b). Closest to such factors are muscle-derived cytokines, called myokines, including IL-6, -8 and -15, which are released upon muscle contraction. These may have immunological roles mediating e.g. exercise-induced anti-inflammatory effects (Nielsen and Pedersen, 2008). In physiological challenges affecting the energy stores, such as 24-hr fasting, strenuous exercise, or ketogenic diet, the skeletal muscle of wild-type mice showed no induction of FGF21. This indicates that FGF21 is a messenger of crisis-level energy depletion in individual skeletal muscle fibers.

FGF21 was recently shown to be secreted from the skeletal muscle upon transgenic over-expression of Akt1, in mice presenting with muscle hypertrophy (Izumiya et al., 2008a). Intriguingly, in the RC-deficient Deletor muscle PI3K/Akt pathway was the most significantly altered pathway. Previously, FGF21 activation has been shown to depend on the nuclear ho none receptor, peroxisome proliferator-activated receptor α (PPARα) in the liver (Badman et al., 2007; Inagaki et al., 2007; Lundasen et al., 2007), and on PPARγ in the adipose tissue (Muise et al., 2008). The inventors did not, however, find indications of PPAR-target up-regulation in the Deletor muscle (data not shown). As Akt regulates mTOR, inhibition of which is known to induce autophagy in nutrient starvation (Schmelzle and Hall, 2000), it is tempting to hypothesize that FGF21 expression in starving muscle is co-regulated with autophagy induction. Furthermore, the RC-deficient muscle fibers have massive proliferation of mitochondria to meet the energy demand. Akt3, which is induced in Deletor muscle (Table S1), has recently been described to regulate mitochondrial proliferation (Wright et al., 2008). These results suggest that the Akt/mTOR pathway in skeletal muscle could be involved in the mitochondrial propagation, induction of mitophagy and the global starvation response through muscle-derived FGF21 signaling.

The only physiological function of FGF21 known to date is to regulate the metabolic adaptation to fasting. Upon limited nutrition, FGF21 is secreted to the circulation by the liver or adipose tissue, and it induces lipolysis in the adipose tissue and ketone body production in the liver of mice (Badman et al., 2007; Inagaki et al., 2007). Sustained FGF21 over-expression resulted in growth hormone resistance (Inagaki et al., 2008). FGF21 has been suggested to be an attractive therapeutic agent for type-2 diabetes, because it lowered efficiently glucose and lipid levels in an insulin-independent manner when administered in diabetic mice and monkeys (Kharitonenkov et al., 2005; Kharitonenkov et al., 2007). Furthermore, FGF21 corrected obesity in mice (Coskun et al., 2008; Xu et al., 2008). The inventors show here that muscle-derived FGF21 induction in RC deficiency replicated all the known global consequences of FGF21 on lipid metabolism, but did not affect growth hormone, insulin or thyroid hormone levels, or insulin or glucose sensitivity. These hormonal effects were, however, seen in mice over-expressing the protein, or after administration of recombinant FGF21, leading to high FGF21 plasma concentrations: liver-specific FGF21 over-expressor mice had 50-170 ng/ml (Kharitonenkov et al., 2005) or >20 ng/ml (Inagaki et al., 2008) of FGF21 in plasma, whereas the Deletors had ˜3 ng/ml. We suggest that FGF21 response may depend on the dose, modest levels specifically mobilizing lipids. Alternatively, the skeletal muscle-derived FGF21 could carry modifications that direct the effect on mobilization of energy stores.

Recent studies have suggested that the metabolic consequences of FGF21 may differ between the species (Galman et al., 2008). Plasma FGF21 levels were increased in obese individuals and in patients with metabolic syndrome (Zhang et al., 2008), and were low in anorexia nervosa (Dostalova et al., 2008). The results obtained by the inventors suggest that skeletal muscle responds to RC dysfunction similarly in mice and men. However, the downstream consequences of FGF21 in mitochondrial disease patients require further attention, as some patients show wasting and cachexia, whereas some have high blood lipids and overweight (Hakonen et al., 2005). The ability of FGF21 to cross the blood-brain barrier (Hsuchou et al., 2007), regulating energy-saving torpor state in rodents (Inagaki et al., 2007), suggests that isolated RC-deficiency in muscle has the potential to induce down-regulation of brain metabolism. Whether FGF21 levels in aging-related COX-deficiency in the muscle can initiate a global ‘energy-saving’ response, inducing lipolysis in adipose tissue and even affect brain metabolism when crossing the blood-brain barrier, is an intriguing thought with wide relevance to human pathology and aging.

Skeletal muscle is the organ with the highest mass in the body, a major user of oxidative ATP, and highly dependent on mitochondrial RC function, as indicated by a plethora of mitochondrial myopathies. The patients often suffer from global metabolic consequences, including muscle atrophy and wasting, cachexia or metabolic syndrome. These symptoms are common for aging-related conditions, which also show declining RC function in the tissues: up to 30% of muscle fibers may be COX-negative in a 90-year-old normal individual (Bua et al., 2006). FGF21, hormone secreted by single RC-deficient muscle fibers, is in a key position in mediating global metabolic changes associated with RC dysfunction in mitochondrial disease and in aging, and offers an attractive pathway to be targeted by therapeutic interventions. Furthermore, it can serve as a novel biomarker for mitochondrial myopathy, potentially reducing the need of invasive muscle samples for disease diagnosis and follow-up.

### FIGURES

FIG. 1. FGF21 is produced by the respiratory chain deficient skeletal muscle and its concentration is increased in Deletor plasma.

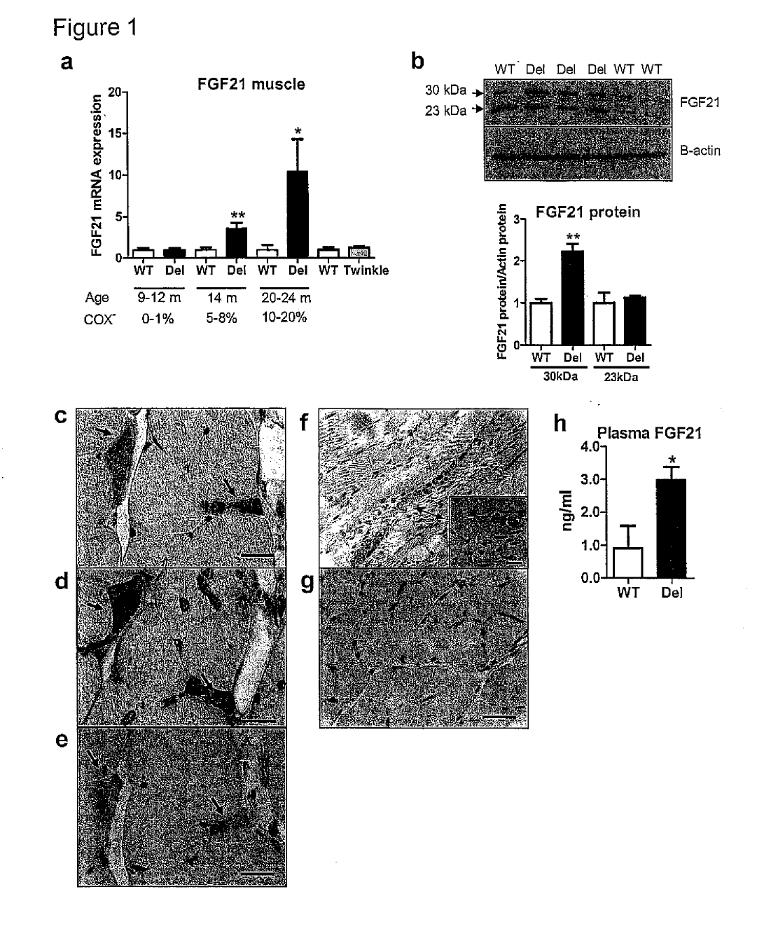

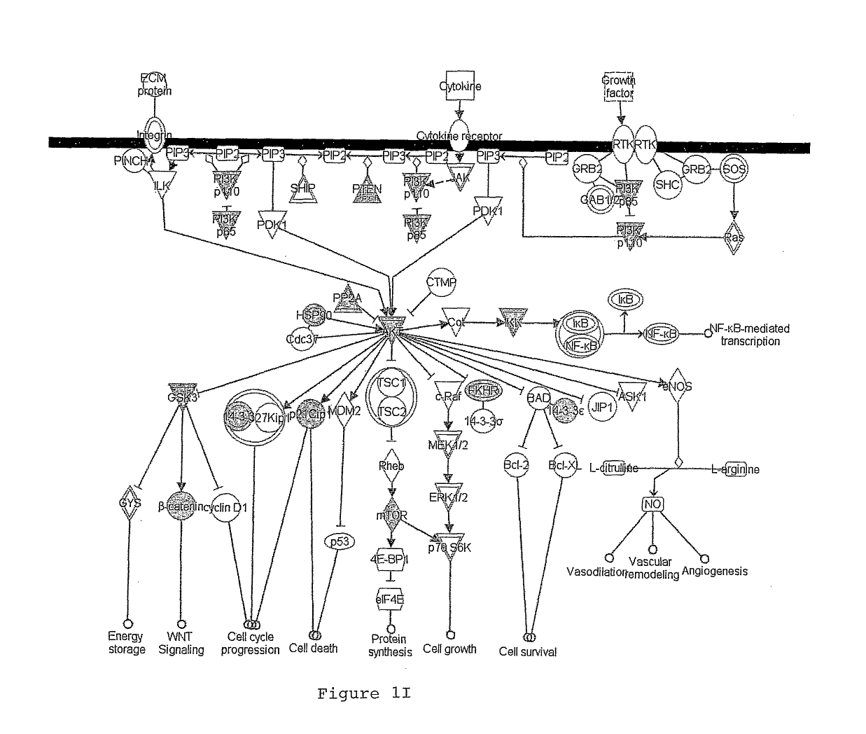

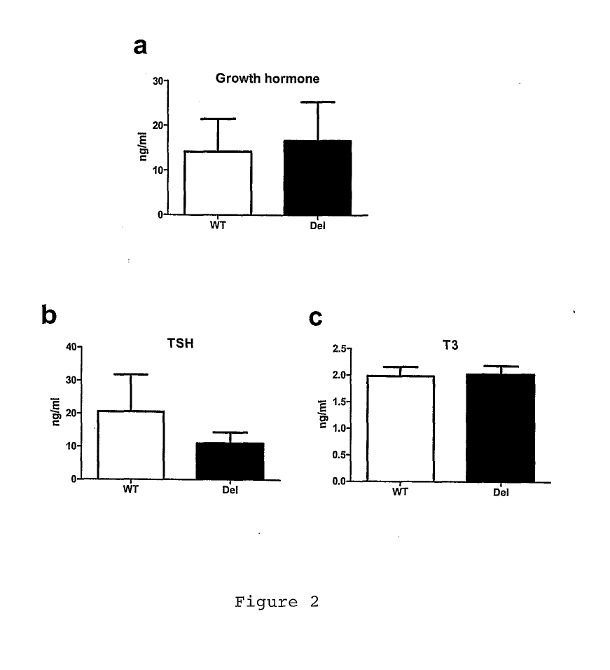

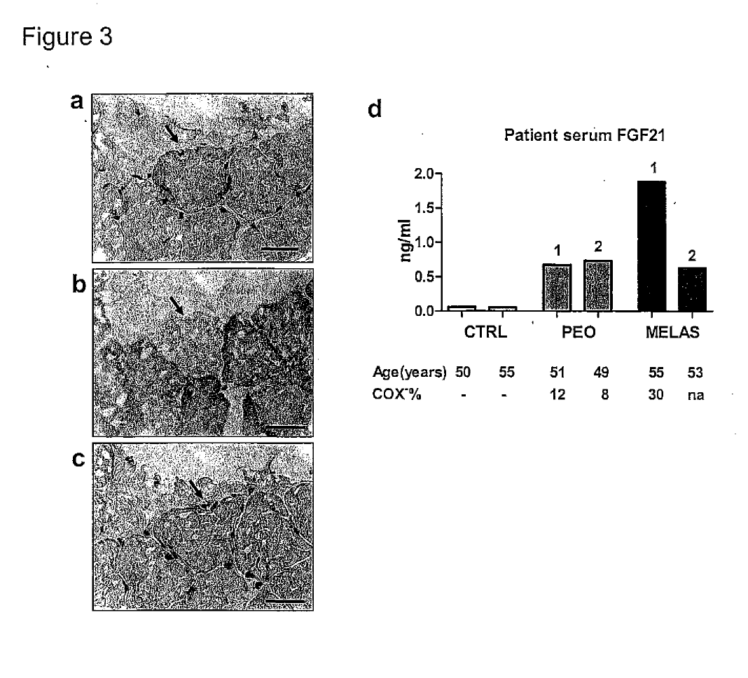

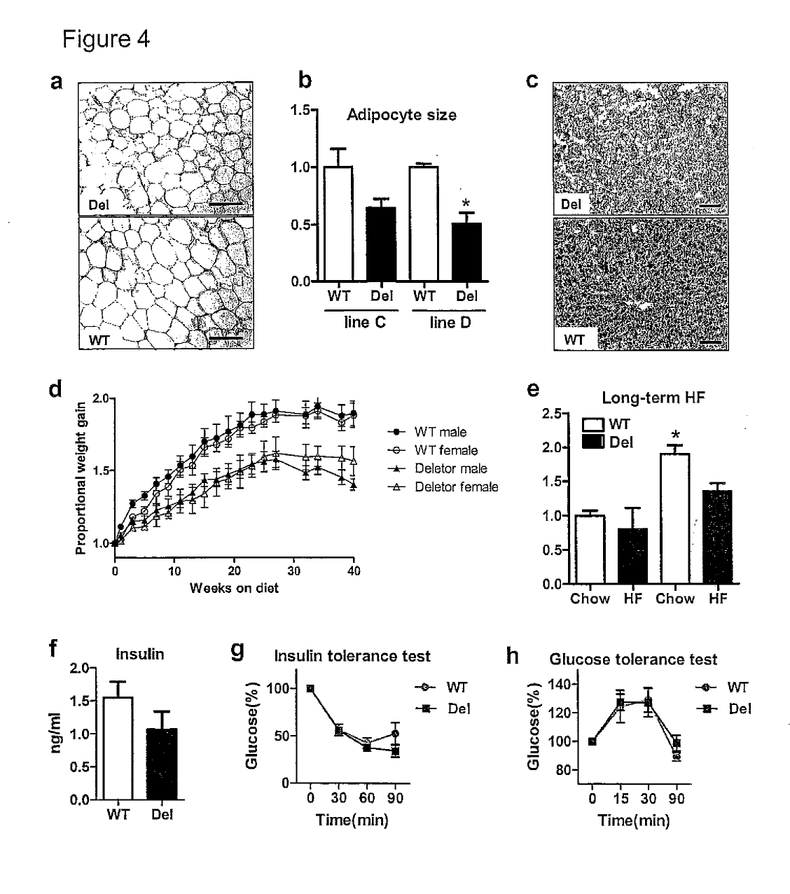

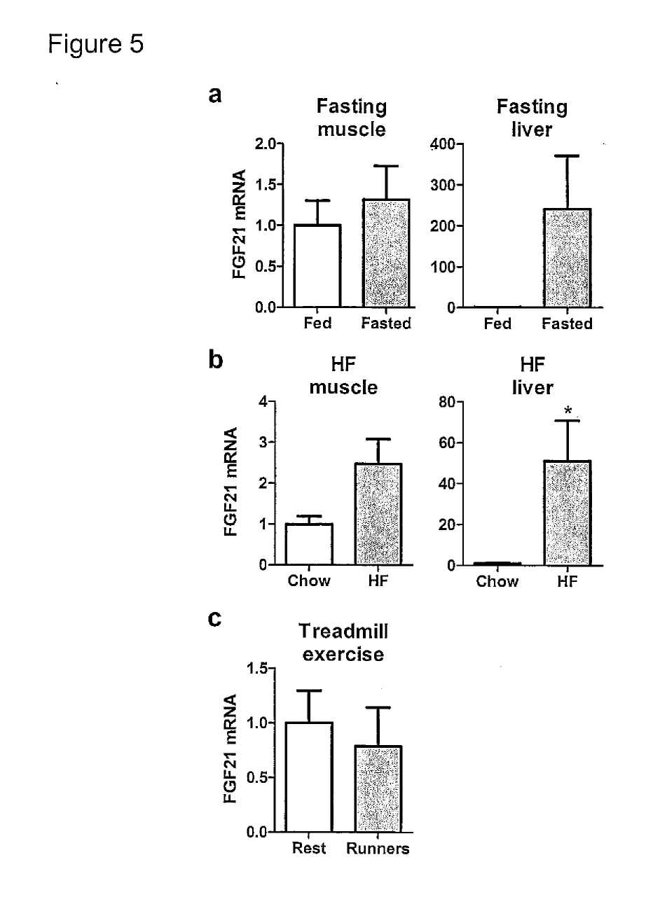

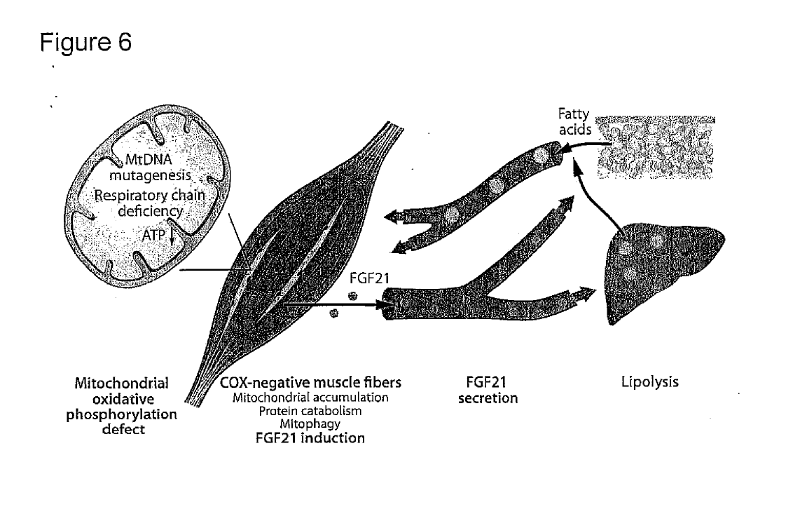

(A) FGF21 mRNA expression levels in Deletor mouse quadriceps femoris muscle follow the progression of the respiratory chain deficiency. In pre-symptomatic age of 9 months, FGF21 expression of Deletor muscle was comparable to that of controls, whereas at 14 months of age, it was 3.6-fold higher (P=0.005) and at 20-24 months of age 10.4-fold higher (P=0.05) than in control littermates (previous data from transgenic C-line; same values for D-line: 1.9-fold induction at 14 months, P=0.0009). Below, the proportion of COX-negative (COX) fibers in the same muscle samples mice. FGF21 mRNA expression in not altered in mice that over-express wild-type Twinkle (P=0.46). The quantitative real-time PCR histogram shows the mean expression levels of 4-5 animals normalised to β-actin. Normalisations with Gapdh and β2-Microglobulin were highly similar (data not shown). M=months.
(B) Western analysis of Deletor muscle FGF21 isoforms. The starvation-associated 30 kDa-isoform of FGF21 is increased in the Deletor muscle (P=0.002) whereas the 23 kDa-isoform remains unchanged. Western blot with FGF21 antibody; n=3.
(C) Deletor mouse skeletal muscle, immunohistochemistry with FGF21 antibody. FGF21 is expressed in those Deletor muscle fibers, which have a massive accumulation of mitochondria: in serial sections identified by
(D) succinate dehydrogenase (complex II, SD70 antibody) immunohistochemistry on paraffin muscle sections. Arrows indicate the diseased muscle fibers.
(E) IgG is the negative control of immunohistochemistry, without primary antibody. Scale bar: 20 μm.
(F) FGF21 specifically localizes to punctuate vesicles around the nuclei and in the cytoplasm as seen in longitudinal paraffin sections of Deletor quadriceps femoris muscle. The inset shows a magnification of the fiber indicated by the arrow. Scale bar: 20 μm.
(G) No FGF21-positivity is identified in the wild-type mouse skeletal muscle. Scale bar: 40 μm.
(H) Plasma FGF21 concentrations are higher in the Deletor (Del) mice compared to age-matched wild-type (WT) mice (P=0.018). n=5 for WT and n=7 for Del. All data are represented as mean±SEM. Two-way Student's t test was used to compare groups.
(I) PI3K/AKT pathway is the most significantly altered pathway in Deletor muscle. Red indicates upregulated transcripts and green down-regulated transcripts.

FIG. 2. Growth hormone and thyroid hormone levels are unaltered in the Deletor mice. (A) Growth hormone concentrations in serum from wild-type (WT) and Deletor (Del) mice of 18-24 months of age. n=9 mice/WT group, n=8 mice/Del group. No significant difference between groups.

(B) Thyroid-stimulating hormone level was measured in Deletor (Del) and wild-type (WT) mouse serum of 18-24 months of age. n=9 mice/WT group, n=7 mice/Del group. p=0.47. No significant difference between groups.
(C) The thyroid hormone T3 level was measured in Deletor (Del) and wild-type (WT) mouse serum of 18-24 months of age. n=9 mice/WT group, n=8 mice/Del group. No significant difference between groups.

All data are represented as mean±SEM. Two-way Student's t-test was used to compare groups.

FIG. 3. FGF21 is induced in the skeletal muscle and serum of patients with mitochondrial myopathy.

(A) Histochemistry of PEO patient's frozen muscle sample: FGF21-antibody shows specific positive staining in those muscle fibers, which are
(B) COX-deficient and have accumulated mitochondria as indicated by the blue SDH-staining. The arrow points to the diseased muscle fiber.
(C) IgG is the negative control of immunohistochemistry, without primary antibody. Scale bar: 20 μm.
(D) Patients with PEO and with MELAS show increased serum concentrations of FGF21. Below is shown the proportion of COX-negative fibers in their skeletal muscle biopsy sample. PEO=progressive external ophthalmoplegia; MELAS=mitochondrial encephalopathy, lactic acidosis and stroke-like episodes; na=not available.

FIG. 4. Metabolic consequences of elevated plasma FGF21 concentration in the Deletor mice.

(A) Hematoxylin & eosin stainings of Deletor and WT mouse white adipose tissue. Notice the small adipocyte size in the Deletor.
(B) Quantification of the adipocyte size. Deletors of both transgenic lines C and D have smaller adipocytes compared to age-matched control mice (P=0.03 in line C; P=0.13 in line D). 185±87 adipocytes/mouse were quantified, at least 3 mice/group. Scale bar: 40 μm.
(C) Oil Red-O staining of Deletor and WT mouse livers. Notice the reduced fat (red droplets) in the Deletor liver. Scale bar: 50 μm.
(D) The Deletor mice gain less weight on a 12-month HF feeding than the control littermates on the same diet. Average proportional weight gain compared to the starting weight of each animal is shown. The values (±SEM) are the average measurements of at least 5 animals in a group. P<0.05 for weeks 1 to 40 for male wild-type versus Deletor males; and for weeks 3 to 40 for female wild-type versus Deletor females. Deletors and wild-type littermates were on HF feeding between the age of 3 and 14 months.
(E) HF diet reduces the level of FGF21 induction in Deletor muscle (n=4 for all groups, P=0.02). Long-term HF feeding did not alter the FGF21 levels of wild-type mice.

The metabolic parameters of Deletor mice were monitored after a moderate 6-h fasting period. (F) For insulin concentrations, sera from wild-type (WT) and Deletor (Del) mice of 18-23 months of age were analyzed. n=9 mice/WT group, n=8 mice/Del group. Insulin levels are not significantly different between WT and Del.

No significant difference in insulin tolerance (G) or glucose tolerance (H) is detected between the WT and the Del (n=5 for both WT and Del).

All data are represented as mean±SEM. Two-way Student's t-test was used to compare groups. *P<0.05. HF=high-fat.

FIG. 5. FGF21 is not induced in skeletal muscle in temporary energy deprivation.

(A) FGF21 is not significantly induced in the skeletal muscle of mice fasted for 24 h (p=0.55), but is highly induced in the livers of the same mice. n=4/group.
(B) HF feeding for four days leads to a potential increase in the level of FGF21 in skeletal muscle (2.5-fold, p=0.06) although the induction is much smaller than in the livers of the same mice. n=4/group.
(C) Strenous treadmill training does not influence the FGF21 expression in skeletal muscle (p=0.66). n=4/group.

FGF21 mRNA expression was determined by quantitative RT-PCR. Data are represented as mean±SEM. Two-way Student's t-test was used to compare groups. *P<0.05.

FIG. 6. Model for FGF21-response of skeletal muscle. Mitochondrial oxidative phosphorylation defect in skeletal muscle leads to FGF21 induction and secretion to circulation. FGF21 mobilizes lipids (yellow droplets) from adipose tissue and liver to provide fuel for the energy-deprived muscle.

### EXPERIMENTAL PROCEDURES

### Patient Samples

The adPEO-patients (males aged 51, 49 and 37 years) were previously described as patients III/4, III/6 and III/9⁹ respectively, and carry the dominant Twinkle dup352-364 mutation (Spelbrink et al., 2001; Suomalainen et al., 1992; Suomalainen et al., 1997). They presently suffer from PEO and generalized moderate muscle weakness. MELAS patient 1 is a woman of 55 years of age, who suffers from mitochondrial myopathy and hypertrophic cardiomyopathy, and has 80% of mutant mtDNA with the MELAS (mitochondrial encephalopathy, lactic acidosis and stroke-like episodes) point mutation A3243G in the quadriceps femoris muscle biopsy sample. Her brother (patient 2) had 18% mutant mtDNA in the leukocytes and had mild cardioventricular hypertrophy. Skeletal muscle biopsy samples (adPEO patients 1-3, MELAS 1) as well as the peripheral venous blood samples processed for serum collection (adPEO-patients 1 and 2, MELAS 1 and 2) were available for the study. The patient studies were approved by the ethical review board of the Department of Neurology, Ophthalmology and Otorhinolaryngology, Helsinki University Central Hospital.

### Mouse Tissue Collection

All animal procedures were performed according to protocols approved by the ethical boards for animal experimentation of National Public Health Institute and Helsinki University, as well as State Provincial offices of Finland (agreement number STU575A/2004) and all experiments were done in accordance with good practice of handling laboratory animals and of genetically modified organisms. The generation and the phenotypes of the Deletor and the wild-type Twinkle overexpressor mice have been previously described (Tyynismaa et al., 2005; Tyynismaa et al., 2004). The transgenic mice were backcrossed to C57B1 background for 10 generations, and the congeneity was confirmed with the Mouse Medium Density SNP Panel (Illumina). The disease phenotype was confirmed to be the same in the C57B1 as in the original FVB/N background. Mice from both backgrounds and from two founder lines (C and D) with different transgene insertion sites were used. The mice were housed in a humidity- and temperature-controlled environment (21° C., 60% humidity, 12-hour light-dark cycle) with free access to chow (Global 18% Protein Rodent Diet, Harlan Teklad) and water. For the high-fat feeding, the mice were maintained between the age of 3 to 14 months on a high-fat D05052004 chow (89.5 kcal % fat, 0.1 carbohydrate, 10.4 proteins; Research Diets Inc.) or control D05052002 chow (11.5 kcal % fat, 78.1 carbohydrate, 10.4 protein). To determine the chow consumption and intestinal fat absorption, daily chow consumption and fecal fat content were measured. Feces were homogenized with scissors and fried in +70° C. for 1 h. 100 mg of dried sample was diluted into 2 ml 2:1 chloroform:methanol and heated in +70° C. for 30 min and then evaporated overnight. The remaining residual material was weighed and assigned as total lipid. For the treadmill exercise, Deletor mice and wild-type littermates were randomized into a treadmill training group (motorized Exer-6M Treadmill, Columbus Instruments), or a sedentary group at the age of 1 year. After initial training of the mice to the test, the mice exercised 5 days a week for 6 weeks. The training speed was calculated from maximal speed and represented an equivalent of 75% of VO2max in 12 month-old male C57BL/6J mice (Schefer and Talan, 1996). The training speed was increased by 1 m/min every week, and in the end of the study, the mice ran until exhaustion. After the tests the mice were sacrificed, and the tissue samples were carefully dissected, snap-frozen in liquid nitrogen and stored at −80° C. until RNA or proteins were extracted.

### RNA Extraction

Total cellular RNA was extracted from frozen tissue samples in TRIzo1 reagent (Invitrogen) and homogenized with Dounce homogenizer with 30 consecutive strokes. Subsequent steps were performed according to the manufacturer's instructions. Qiagen RNeasy Mini Kit was used to clean up the total RNA after the TRIzo1 protocol. The quality of RNA was tested by analyzing the intensities and ratios of cytoplasmic ribosomal RNAs 18S and 28S with 2100 Bioanalyzer (Agilent Technologies).

### Gene Expression Profiling

Gene expression profiles were determined from quadriceps femoris of three female Deletor and four female control littermate mice, aged 21-24 months, using the GeneChip Mouse Genome 430 2.0 array (Affymetrix). One microarray experiment contained RNA from a single mouse. Labelling, hybridization, and scanning were performed according to manufacturer's instructions. Data was normalized according to the Robust Multichip Average procedure, RMA (Irizarry et al., 2003) using the Genespring 7.2 software. In order to determine those genes that were differentially expressed between transgenic and control mice, a t-test was applied. The transcripts that filled the p-value criterion of <0.05 for differential expression between transgenic and control mice were selected for further analysis regardless of fold change. The false-positive rate (FDR) was determined as in (Zapala et al., 2005) to be 0.67%. The selected transcripts were subjected to pathway analysis using the Ingenuity Pathway Analysis (Ingenuity Systems), a literature mining cured database.

### Reverse Transcription PCR and Quantitative PCR

One microgram of total RNA was used to generate cDNA using random hexamers with Moloney Murine Leukemia Virus Reverse Transcriptase (Promega). Quantitative real-time PCRs were performed on cDNA with DyNAmo Flash SYBR Green qPCR Kit (Finnzymes) on an ABI prism 7000 light cycler. Gapdh, β2-microglobulin and β-actin were used as control genes. The primer sequences are available on request.

### Histochemistry

The mouse quadriceps femoris muscle samples were fixed in 10% buffered formalin and embedded in paraffin. The primary antibodies used were monoclonal anti-CII-Fp (MS204, Molecular Probes, working dilution 1:200) and goat anti-mouse FGF21 antibody (AF3057, R&D Systems, 1:1,000). The samples were blocked for non-specific staining by incubating for 30 minutes in 2% horse or rabbit serum, respectively. Further detection was carried out with the Vectastain ABC Mouse or Goat IgG Kits (Vector Laboratories) according to the manufacturer's instructions followed by chromogen DAB staining. The slides were briefly counterstained with hematoxylin. Light microscopy was performed with Axioplan 2 (Carl Zeiss).

The human skeletal muscle samples were frozen in isopentane/liquid nitrogen. Cryostat sections were stained simultaneously for cytochrome c oxidase (COX) and succinate dehydrogenase (SDH) activities. For immunohistochemistry, frozen sections were fixed with acetone and blocked in 2% horse serum. The primary antibody was monoclonal anti-human FGF21 antibody (MAB2537, R&D Systems, 5 μg/ml) diluted in Dako REAL Antibody Diluent (Dako). Further detection was carried out with the Vectastain ABC Mouse IgG Kit (Vector Laboratories) and DAB staining.

For the adipocyte size quantification, formalin-fixed paraffin sections were H&E stained and studied under Axioplan 2 microscope. The size of adipocytes (on average 185±87 cells per mouse) was quantified with the ImageJ program (http://rsbweb.nih.gov/ij/index.html).

The Oil Red O staining was performed on 8 μm frozen tissue sections from liver. The slides were fixed in formic calcium (1:10:1 concentrated formalin: aqua: 10% calcium chloride) for 5 minutes. Slides were then incubated in Oil Red 0 solution for 10 min and the nuclei were stained with Mayer's haematoxylin for 2 min. Oil Red 0 stock solution (1 mg/ml in isopropanol) was made by heating the reagent and alcohol in 56° C. for 30 min. Fresh working solution was made from stock solution and aqua (3:2). Solution was mixed well, let to stand for 5 minutes, filtrated and used within few hours.

### Quantitative Western Blot Analysis

The proteins from muscle samples were extracted by homogenizing in RIPA buffer (0.75M NaCl, 5% NP40, 2.5% Sodium deoxycholate, 0.5% SDS, 0.25M Tris-HCl pH 8.0, 10 mM DTT, protease inhibitors). The samples were incubated on ice for 20 min and centrifuged at 13000 g for 5 min. The supernatant was collected and stored in −80° C. Protein samples were separated on 15% SDS-PAGE gels and transferred to Immobilon-FL transfer membrane (Millipore) using the Bio-Rad Western Transfer unit. The membranes were blocked in 1% BSA (for FGF21) or 5% milk (for Actin) in TBS-Tween20 (0.1%) for 1 hour at room temperature. Blocking solutions were replaced with fresh solution and antisera at the appropriate dilution. The primary antibodies used were goat anti-mouse FGF21 antibody (AF3057, R&D Systems, 1:1,000) and goat polyclonal Actin (sc-1616, Santa Cruz Biotechnology, 1:1,000). Blots were incubated at 4° C. overnight. The membranes were washed 3 times with TBS+0.1% Tween20 for a total of 15 minutes. Secondary anti-goat IgG antibody (Calbiochem) was incubated with the membranes at a dilution of 1:10,000 in blocking buffer at room temperature for 1 h. The secondary antibody was removed and filters washed three times in TBS+0.1% Tween20 for a total of 15 minutes. ECL Plus Western Blotting Detection System (GE Healthcare) was used for the detection of signals with Typhoon 9400 (Amersham Biosciences) and quantified with the ImageQuant v5.0 software.

### Metabolic Measurements

The plasma and serum samples were prepared by centrifugation after blood collection and stored at −80° C. until analyzed. Mouse plasma FGF21 concentrations were measured using a radioimmunoassay kit (Phoenix Pharmaceuticals, Inc). Human serum concentrations were measured by ELISA kit (BioVendor). Serum growth hormone and insulin concentrations were determined using ELISA kits for mouse/rat (LINCO Research, Millipore). Serum T3 concentrations were determined using ELISA kit for mouse/rat (Calbiotech) and TSH using ELISA kit for mouse/rat (Gentaur). For insulin tolerance test (ITT), mice were fasted 6 hours and challenged by i.p. insulin injection of 1 U/kg (NovoRapid, Novo Nordisk A/S). Blood samples were taken from tail vein and blood glucose levels were measured using Precision Xceed glucometer (Abbot Laboratories) immediately prior to insulin administration, and at 30, 60 and 90 minutes after insulin administration. For oral glucose tolerance test (OGTT), mice were fasted 6 hours and challenged by an oral glucose load (2 g/kg). Blood glucose levels were measured prior to glucose administration, and at 15, 30, and 90 minutes after glucose administration.

### Statistical Analysis

Statistical analysis was performed using the Student's t-test, with two-tailed P-values. Data were expressed as mean±SEM.

### REFERENCES

Badman, M. K., Pissios, P., Kennedy, A. R., Koukos, G., Flier, J. S., and Maratos-Flier, E. (2007). Hepatic fibroblast growth factor 21 is regulated by PPARalpha and is a key mediator of hepatic lipid metabolism in ketotic states. Cell Metab 5, 426-437.
Barros, M. H., and Tzagoloff, A. (2002). Regulation of the heme A biosynthetic pathway in Saccharomyces cerevisiae. FEBS Lett 516, 119-123.
Bua, E., Johnson, S., Herbst, A., Delong, B., McKenzie, D., Salamat, S., and Aiken, J. M. (2006). Mitochondrial DNA-deletion mutations accumulate intracellularly to detrimental levels in aged human skeletal muscle fibers. Am J Hum Genet 79, 469-480.
Coskun, T., Bina, H. A., Schneider, M. A., Dunbar, J. D., Hu, C. C., Chen, Y., Moller, D. E., and Kharitonenkov, A. (2008). FGF21 Corrects Obesity in Mice. Endocrinology 149, 6018-6027.
Diaz, F., Thomas, C. K., Garcia, S., Hernandez, D., and Moraes, C. T. (2005). Mice lacking COX10 in skeletal muscle recapitulate the phenotype of progressive mitochondrial myopathies associated with cytochrome c oxidase deficiency. Rum Mol Genet 14, 2737-2748.
Dostalova, I., Kavalkova, P., Haluzikova, D., Lacinova, Z., Mraz, M., Papezova, H., and Haluzik, M. (2008). Plasma concentrations of fibroblast growth factors 19 and 21 in patients with anorexia nervosa. J Clin Endocrinol Metab 93, 3627-3632.
Galman, C., Lundasen, T., Kharitonenkov, A., Bina, H. A., Eriksson, M., Hafstrom, I., Dahlin, M., Amark, P., Angelin, B., and Rudling, M. (2008). The circulating metabolic regulator FGF21 is induced by prolonged fasting and PPARalpha activation in man. Cell Metab 8, 169-174.
Hakonen, A. H., Heiskanen, S., Juvonen, V., Lappalainen, I., Luoma, P. T., Rantamaki, M., Goethem, G. V., Lofgren, A., Hackman, P., Paetau, A., et al. (2005). Mitochondrial DNA polymerase W748S mutation: a common cause of autosomal recessive ataxia with ancient European origin. Am J Hum Genet 77, 430-441.
Hsuchou, H., Pan, W., and Kastin, A. J. (2007). The fasting polypeptide FGF21 can enter brain from blood. Peptides 28, 2382-2386.
Inagaki, T., Dutchak, P., Zhao, G., Ding, X., Gautron, L., Parameswara, V., Li, Y., Goetz, R., Mohammadi, M., Esser, V., et al. (2007). Endocrine regulation of the fasting response by PPARalpha-mediated induction of fibroblast growth factor 21. Cell Metab 5, 415-425.
Inagaki, T., Lin, V. Y., Goetz, R., Mohammadi, M., Mangelsdorf, D. J., and Kliewer, S. A. (2008). Inhibition of Growth Hormone Signaling by the Fasting-Induced Hormone FGF21. Cell Metab 8, 77-83.
Irizarry, R. A., Bolstad, B. M., Collin, F., Cope, L. M., Hobbs, B., and Speed, T. P. (2003). Summaries of Affymetrix GeneChip probe level data. Nucleic Acids Res 31, e15.
Izumiya, Y., Bina, H. A., Ouchi, N., Akasaki, Y., Kharitonenkov, A., and Walsh, K. (2008a). FGF21 is an Akt-regulated myokine. FEBS Lett 582, 3805-3810.
Izumiya, Y., Hopkins, T., Morris, C., Sato, K., Zeng, L., Viereck, J., Hamilton, J. A., Ouchi, N., LeBrasseur, N. K., and Walsh, K. (2008b). Fast/Glycolytic muscle fiber growth reduces fat mass and improves metabolic parameters in obese mice. Cell Metab 7, 159-172.
Kaukonen, J., Juselius, J. K., Tiranti, V., Kyttala, A., Zeviani, M., Comi, G. P., Keranen, S., Peltonen, L., and Suomalainen, A. (2000). Role of adenine nucleotide translocator 1 in mtDNA maintenance. Science 289, 782-785.
Kharitonenkov, A., and Shanafelt, A. B. (2008). Fibroblast growth factor-21 as a therapeutic agent for metabolic diseases. BioDrugs 22, 37-44.
Kharitonenkov, A., Shiyanova, T. L., Koester, A., Ford, A. M., Micanovic, R., Galbreath, E. J., Sandusky, G. E., Hammond, L. J., Moyers, J. S., Owens, R. A., et al. (2005). FGF-21 as a novel metabolic regulator. J Clin Invest 115, 1627-1635.
Kharitonenkov, A., Wroblewski, V. J., Koester, A., Chen, Y. F., Clutinger, C. K., Tigno, X. T., Hansen, B. C., Shanafelt, A. B., and Etgen, G. J. (2007). The metabolic state of diabetic monkeys is regulated by fibroblast growth factor-21. Endocrinology 148, 774-781.
Lauritzen, H. P., Galbo, H., Brandauer, J., Goodyear, L. J., and Ploug, T. (2008). Large GLUT4 vesicles are stationary while locally and reversibly depleted during transient insulin stimulation of skeletal muscle of living mice: imaging analysis of GLUT4-enhanced green fluorescent protein vesicle dynamics. Diabetes 57, 315-324.
Longley, M. J., Clark, S., Yu Wai Man, C., Hudson, G., Durham, S. E., Taylor, R. W., Nightingale, S., Turnbull, D. M., Copeland, W. C., and Chinnery, P. F. (2006). Mutant POLG2 disrupts DNA polymerase gamma subunits and causes progressive external ophthalmoplegia. Am J Hum Genet 78, 1026-1034.
Lundasen, T., Hunt, M. C., Nilsson, L. M., Sanyal, S., Angelin, B., Alexson, S. E., and Rudling, M. (2007). PPARalpha is a key regulator of hepatic FGF21. Biochem Biophys Res Commun 360, 437-440.
Muise, E. S., Azzolina, B., Kuo, D. W., El-Sherbeini, M., Tan, Y., Yuan, X., Mu, J., Thompson, J. R., Berger, J. P., and Wong, K. K. (2008). Adipose Fibroblast Growth Factor 21 is Up-Regulated by PPAR{gamma} and Altered Metabolic states. Mol Pharmacol 74, 403-412.
Nielsen, S., and Pedersen, B. K. (2008). Skeletal muscle as an immunogenic organ. Curr Opin Pharmacol 8, 346-351.
Oldfors, A., Moslemi, A. R., Jonasson, L., Ohlsson, M., Kollberg, G., and Lindberg, C. (2006). Mitochondrial abnormalities in inclusion-body myositis. Neurology 66, 549-55.
Reitman, M. L. (2007). FGF21: a missing link in the biology of fasting. Cell Metab 5, 405-407.
Schefer, V., and Talan, M. I. (1996). Oxygen consumption in adult and AGED C57BL/6J mice during acute treadmill exercise of different intensity. Exp Gerontol 31, 387-392.
Schmelzle, T., and Hall, M. N. (2000). TOR, a central controller of cell growth. Cell 103, 253-262.
Spelbrink, J. N., Li, F. Y., Tiranti, V., Nikali, K., Yuan, Q. P., Tariq, M., Wanrooij, S., Garrido, N., Comi, G., Morandi, L., et al. (2001). Human mitochondrial DNA deletions associated with mutations in the gene encoding Twinkle, a phage T7 gene 4-like protein localized in mitochondria. Nat Genet 28, 223-231.
Suomalainen, A., Majander, A., Haltia, M., Somer, H., Lonnqvist, J., Savontaus, M. L., and Peltonen, L. (1992). Multiple deletions of mitochondrial DNA in several tissues of a patient with severe retarded depression and familial progressive external ophthalmoplegia. J Clin Invest 90, 61-66.
Suomalainen, A., Majander, A., Wallin, M., Setala, K., Kontula, K., Leinonen, H., Salmi, T., Paetau, A., Haltia, M., Valanne, L., et al. (1997). Autosomal dominant progressive external ophthalmoplegia with multiple deletions of mtDNA: clinical, biochemical, and molecular genetic features of the 10q-linked disease. Neurology 48, 1244-1253.
Trifunovic, A., Wredenberg, A., Falkenberg, M., Spelbrink, J. N., Rovio, A. T., Bruder, C. E., Bohlooly, Y. M., Gidlof, S., Oldfors, A., Wibom, R, et al. (2004). Premature ageing in mice expressing defective mitochondrial DNA polymerase. Nature 429, 417-423.
Tyynismaa, H., Mjosund, K. P., Wanrooij, S., Lappalainen, I., Ylikallio, E., Jalanko, A., Spelbrink, J. N., Paetau, A., and Suomalainen, A. (2005). Mutant mitochondrial helicase Twinkle causes multiple mtDNA deletions and a late-onset mitochondrial disease in mice. Proc Natl Acad Sci USA 102, 17687-17692.
Tyynismaa, H., Sembongi, H., Bokori-Brown, M., Granycome, C., Ashley, N., Poulton, J., Jalanko, A., Spelbrink, J. N., Holt, I. J., and Suomalainen, A. (2004). Twinkle helicase is essential for mtDNA maintenance and regulates mtDNA copy number. Hum Mol Genet 13, 3219-3227.
Van Goethem, G., Dermaut, B., Lofgren, A., Martin, J. J., and Van Broeckhoven, C. (2001). Mutation of POLG is associated with progressive external ophthalmoplegia characterized by mtDNA deletions. Nat Genet 28, 211-212.
Wenz, T., Diaz, F., Spiegelman, B. M., and Moraes, C. T. (2008). Activation of the PPAR/PGC-1alpha pathway prevents a bioenergetic deficit and effectively improves a mitochondrial myopathy phenotype. Cell Metab 8, 249-256.
Wright, G. L., Maroulakou, I. G., Eldridge, J., Liby, T. L., Sridharan, V., Tsichlis, P. N., and Muise-Helmericks, R. C. (2008). VEGF stimulation of mitochondrial biogenesis: requirement of AKT3 kinase. Faseb J 22, 3264-3275.
Xu, J., Lloyd, D. J., Hale, C., Stanislaus, S., Chen, M., Sivits, G., Vonderfecht, S. N., Hecht, R., Li, L., Lindberg, R. A., et al. (2008). FGF21 Reverses Hepatic Steatosis, Increases Energy Expenditure and Improves Insulin Sensitivity in Diet-induced Obese Mice. Diabetes 58, 250-259.
Zapala, M. A., Hovatta, I., Ellison, J. A., Wodicka, L., Del Rio, J. A., Tennant, R., Tynan, W., Broide, R. S., Helton, R., Stoveken, B. S., et al. (2005). Adult mouse brain gene expression patterns bear an embryologic imprint. Proc Natl Acad Sci U S A 102, 10357-10362.
Zeviani, M., Servidei, S., Gellera, C., Bertini, E., DiMauro, S., and DiDonato, S. (1989). An autosomal dominant disorder with multiple deletions of mitochondrial DNA starting at the D-loop region. Nature 339, 309-311.
Zhang, X., Yeung, D. C., Karpisek, M., Stejskal, D., Zhou, Z. G., Liu, F., Wong, R. L., Chow, W. S., Tso, A. W., Lam, K. S., and Xu, A. (2008). Serum FGF21 levels are increased in obesity and are independently associated with the metabolic syndrome in humans. Diabetes 57, 1246-1253.

1. A method of assessing whether a subject is affected with or at risk for developing a dysfunction of the mitochondrial respiratory chain, the method comprising determining the concentration of fibroblast growth factor 21 (FGF21) in a biological sample from the subject, and comparing it to the concentration of FGF21 in a biological sample from at least one normal control, wherein an increase in the concentration of FGF21 in the biological sample from the subject when compared to the concentration of FGF21 in the biological sample from at least one normal control is indicative of occurrence of the dysfunction of the mitochondrial respiratory chain in said subject, or of risk for developing said dysfunction.

2. A method according to claim 1, wherein the dysfunction of the mitochondrial respiratory chain is of a type manifesting with muscle-related or brain-related or heart related or liver-related symptoms.

3. A method according to claim 1, wherein the dysfunction of the mitochondrial respiratory chain is of a type previously known to involve cytochrome-c-oxidase negative muscle fibers.

4. A method according to claim 1, wherein the biological sample is selected from the group consisting of blood, serum, plasma, urine, cerebrospinal fluid, and other bodily fluids.

5. A method according to claim 1, wherein the concentration of FGF21 in the biological sample is determined by a detection method selected from the group consisting of ELISA, RIA and other immuno-detection based methods, other methods based on direct protein identification such as mass spectrometric analysis, and any other suitable detection methods.

6. A method according to claim 1, wherein the extent of the increase in said concentration of FGF21 as compared to the normal control correlates with the severity of the mitochondrial alterations in the muscles of said subject.

7. A method for follow-up of a dysfunction of the mitochondrial respiratory chain in a subject diagnosed with but not being treated for said dysfunction, the method comprising the steps of:
a. obtaining a biological sample from said subject
b. determining the concentration of fibroblast growth factor 21 (FGF21) in the biological sample; and
c. evaluating the concentration of FGF21, wherein an increase in the concentration of FGF21 in the biological sample from said subject when compared to the concentration of FGF21 in a biological sample taken from the same subject at an earlier time point is indicative of a progression of said dysfunction, whereas a decrease is indicative of a regression of said dysfunction.

8. A method according to claim 7, wherein the dysfunction of the mitochondrial respiratory chain is of a type manifesting with muscle-related or brain-related or heart related or liver-related symptoms.

9. A method according to claim 7, wherein the dysfunction of the mitochondrial respiratory chain is of a type previously known to involve cytochrome-c-oxidase negative muscle fibers.

10. A method according to claim 7, wherein the biological sample is selected from the group consisting of blood, serum, plasma, urine, cerebrospinal fluid, and other bodily fluids.

11. A method according to claim 7, wherein the concentration of FGF21 in the biological sample is determined by a detection method selected from the group consisting of ELISA, RIA and other immuno-detection based methods, other methods based on direct protein identification such as mass spectrometric analysis, and any other suitable detection methods.

12. A method for treating a subject having a dysfunction of the mitochondrial respiratory chain, wherein an agent inhibiting fibroblast growth factor 21 (FGF21) or inhibiting its receptor is administered to said subject.

13. A method according to claim 12, wherein said agent inhibiting FGF21 or inhibiting its receptor is an antibody or a chemical inhibitor compound.

14. A method according to claim 12, wherein the dysfunction of the mitochondrial respiratory chain is of a type manifesting with muscle-related or brain-related or heart related or liver-related symptoms.

15. A method according to claim 12, wherein the dysfunction of the mitochondrial respiratory chain is of a type previously known to involve cytochrome-c-oxidase negative muscle fibers.

16. A method for treating a subject having a dysfunction of the mitochondrial respiratory chain, wherein an agent mimicking or stimulating fibroblast growth factor 21 (FGF21) or its receptor, or in vitro produced recombinant FGF21, is administered to said subject.

17. A method according to claim 16, wherein the dysfunction of the mitochondrial respiratory chain is of a type manifesting with muscle-related or brain-related or heart related or liver-related symptoms.

18. A method according to claim 16, wherein the dysfunction of the mitochondrial respiratory chain is of a type previously known to involve cytochrome-c-oxidase negative muscle fibers.

19. A method for determining whether a dysfunction of the mitochondrial respiratory chain in a subject diagnosed with and being treated for said dysfunction is responding to the treatment, and for selecting patients for clinical trials, the method comprising the steps of:
a. obtaining a biological sample from said subject;
b. determining the concentration of fibroblast growth factor 21 (FGF21) in the biological sample; and
c. evaluating the concentration of FGF21, wherein a decrease in the concentration of FGF21 in the biological sample from said subject when compared to the concentration of FGF21 in a biological sample taken from the same subject at an earlier time point is indicative of a positive response to the treatment, whereas an increase is indicative of a negative response to the treatment or non-responsiveness to the treatment.

20. A method according to claim 19, wherein the dysfunction of the mitochondrial respiratory chain is of a type manifesting with muscle-related or brain-related or heart related or liver-related symptoms.

21. A method according to claim 19, wherein the dysfunction of the mitochondrial respiratory chain is of a type previously known to involve cytochrome-c-oxidase negative muscle fibers.

22. A method according to claim 19, wherein the biological sample is selected from the group consisting of blood, serum, plasma, urine, cerebrospinal fluid, and other bodily fluids.

23. A method according to claim 19, wherein the concentration of FGF21 in the biological sample is determined by a detection method selected from the group consisting of ELISA, RIA and other immuno-detection based methods, other methods based on direct protein identification such as mass spectrometric analysis, and any other suitable detection methods.

24. A method according to claim 19, wherein the extent of the decrease in said concentration of FGF21 as compared to the concentration at earlier time point correlates with the effectiveness of the treatment.
